(19) United States
(12) Patent Application Publication
(10) Pub. No.: US 2026/0086026 A1
(43) Pub. Date: Mar. 26, 2026

(54) BREATH SENSOR AND BREATH MEASUREMENT METHOD

(71) Applicant: Asahi Kasei Microdevices Corporation, Tokyo (JP)

(72) Inventors: Yuta TAKAGI, Tokyo (JP); Seiichi TOKUO, Tokyo (JP)

(21) Appl. No.: 19/339,257

(22) Filed: Sep. 24, 2025

(30) Foreign Application Priority Data
Sep. 25, 2024 (JP) 2024-166668
Sep. 11, 2025 (JP) 2025-151573

(51) Int. Cl. G01N 21/3504; G01N 33/497

(52) U.S. Cl. CPC G01N 21/3504; G01N 33/4972; G01N 2201/06186

(57) ABSTRACT

A potential difference between a first terminal and a second terminal of a light emitting element is maintained at a first value or more in a first measurement period, and is maintained at 0V or more and less than the first value in a third measurement period, a concentration of the measurement target gas is measured based on a second intensity of the light received by the light receiving element during a second measurement period which starts at a second timing that is later than a first timing at which the first measurement period starts and before a third timing at which the third measurement period starts and a fourth intensity of the light received by the light receiving element during a fourth measurement period which starts at a fourth timing that is later than the third timing and before a next occurrence of the first timing.

The contents of the following patent application(s) are incorporated herein by reference:
NO. 2024-166668 filed in JP on Sep. 25, 2024 and
NO. 2025-151573 filed in JP on Sep. 11, 2025.

### BACKGROUND

### 1. Technical Field

The present invention relates to a breath sensor and a breath measurement method.

### 2. Related Art

Patent document 1 describes that “The invention herein described relates to a sensor and method for measuring the concentration of alcohol in alcohol-hydrocarbon mixtures” (Technical Field).

### RELATED ART DOCUMENTS

### Patent Document

Patent Document 1: Japanese translation publication of a PCT route patent application No. H4-501769

### BRIEF DESCRIPTION OF THE DRAWINGS

FIG. 1A illustrates an example of a gas sensor 100 according to one embodiment of the present invention.

FIG. 1B illustrates another example of the gas sensor 100 according to one embodiment of the present invention.

FIG. 2 is a circuit diagram illustrating an example of a connection relationship between a light emitting element 10 and a power supply unit 20 in the gas sensor 100 of FIG. 1A or FIG. 1B.

FIG. 3 is a circuit diagram illustrating another example of the connection relationship between the light emitting element 10 and the power supply unit 20 in the gas sensor 100 of FIG. 1A or FIG. 1B.

FIG. 4 illustrates an example of a relationship between a polarity of a direct current power and a time instant t.

FIG. 5 illustrates another example of the relationship between the polarity of the direct current power and the time instant t.

FIG. 6 illustrates another example of the relationship between the polarity of the direct current power and the time instant t.

FIG. 7 illustrates an example of a measurement target 200.

FIG. 8 illustrates an example of a relationship between a potential V of a second terminal Ev2 and a time instant t.

FIG. 9 illustrates an example of a relationship between a current I that flows through a filament 14 and a time instant t.

FIG. 10 illustrates a comparative example of the relationship between the potential V of the second terminal Ev2 and the time instant t.

FIG. 11 illustrates a comparative example of the relationship between the current I that flows through the filament 14 and the time instant t.

FIG. 12 illustrates another example of the relationship between the potential V of the second terminal Ev2 and the time instant t.

FIG. 13 illustrates of an example of a relationship between a current I that flows through the filament 14 and a time instant t.

FIG. 14 illustrates another example of the relationship between the polarity of the direct current power and the time instant t.

FIG. 15 illustrates another example of the relationship between the polarity of the direct current power and the time instant t.

FIG. 16 illustrates another example of the relationship between the polarity of the direct current power and the time instant t.

FIG. 17 illustrates an example of a relationship between an intensity Ir of light 12 and a concentration of the measurement target gas 90.

FIG. 18 illustrates another example of the relationship between the polarity of the direct current power and the time instant t.

FIG. 19 illustrates an example of a relationship between an intensity of the light 12 received by a first light receiving element 52 and a time instant t.

FIG. 20 illustrates an example of a power supply system 300 according to one embodiment of the present invention.

FIG. 21 is a flowchart illustrating an example of a measurement method of the gas concentration according to one embodiment of the present invention.

### DESCRIPTION OF EXEMPLARY EMBODIMENTS

Hereinafter, the present invention will be described through embodiments of the invention, but the following embodiments do not limit the invention according to the claims. In addition, not all combinations of features described in the embodiments are essential to a solution of the invention.

In the present specification, the arrangement of each element may be described as, for each element of a circuit, a third element is arranged between a first element and a second element. Such description for the arrangement refers to a position of each element in an electric route, and does not limit a position of each element in a space.

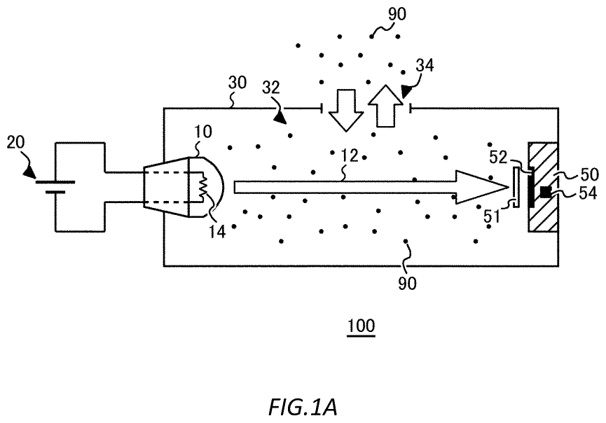

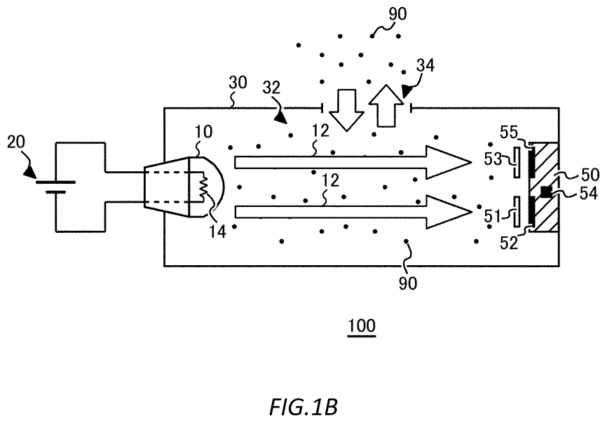

FIG. 1A illustrates an example of a gas sensor 100 according to one embodiment of the present invention. The gas sensor 100 measures a concentration of a measurement target gas 90. The measurement target gas 90 is gas related to breath as an example, but the measurement target gas 90 is not limited thereto. The gas related to breath is gas including gas included in breath. The gas related to breath may be a part of gas included in the breath, may be the entire gas, or may be gas in which these types of gas and another gas are mixed. For example, the gas sensor 100 may be a breath sensor that measures a carbon dioxide concentration or an alcoholic concentration or the like in the breath, but the measurement target gas 90 is not limited thereto. The measurement target gas 90 is, for example, carbon dioxide, water vapor, methane, ethane, propane, butane, formaldehyde, carbon monoxide, nitrogen monoxide, ammonium, sulfur dioxide, alcohol (methanol, ethanol, or the like), chlorofluorocarbon, hydrochlorofluorocarbon, hydrofluorocarbon, refrigerant gas (R32, R290, or the like), or the like, or mixed gas of the above.

The concentration of the gas is, for example, a volume concentration (vol %). T gas sensor 100 includes a light emitting element 10, a power supply unit 20, a cell 30, and a concentration measurement unit 50. The gas sensor 100 may include a first optical filter 51. The measurement target gas 90 is contained in an inner space 32 of the cell 30. The cell 30 may have a gas inlet/outlet 34. The measurement target gas 90 enters the inner space 32 from the gas inlet/outlet 34, and moves to the outside of the cell 30 from the inner space 32. The inlet through which the measurement target gas 90 enters the inner space 32 and the outlet through which it moves out from the inner space 32 may be a common opening such as illustrated in FIG. 1A, or may be separate openings. The gas sensor 100 may measure the concentration of the measurement target gas 90 that passes through the cell 30, or may measure the concentration of the measurement target gas 90 that is encapsulated in the cell 30.

The light emitting element 10 emits light 12 to be radiated to the measurement target gas 90. The light emitting element 10 radiates the light 12 to the measurement target gas 90 contained in the inner space 32. The light emitting element 10 has a filament 14. The filament 14 may be formed mainly of W (tungsten). The filament 14 may be W (tungsten) to which ThO₂ (thorium oxide) or an oxide of a rare earth element is added. The power supply unit 20 supplies direct current power to the light emitting element 10. The light emitting element 10 is of a thermal radiation type. By supplying the direct current power to the light emitting element 10, the filament 14 is heated. The filament 14 is heated, and thereby the light emitting element 10 emits the light 12 with a predetermined wavelength. The light 12 may be an infrared light (wavelength of 780 nm or more and 15000 nm or less), may be a visible light (wavelength of 380 nm or more and 780 nm or less), or may be an ultraviolet light (wavelength of 100 nm or more and 380 nm or less). The light 12 that is radiated to the measurement target gas 90 and passed through the measurement target gas 90 is incident on the concentration measurement unit 50.

The concentration measurement unit 50 measures a concentration of the measurement target gas 90. The concentration measurement unit 50 may have a first light receiving element 52 that receives the light 12 radiated to the measurement target gas 90. The first optical filter 51 may be provided between the light emitting element 10 and the first light receiving element 52 in the optical path of the light 12. The first optical filter 51 is a filter that transmits the light 12 in a wavelength band which is absorbed by the measurement target gas 90. The first light receiving element 52 may receive the light 12 that transmitted through the first optical filter 51.

The concentration measurement unit 50 may measure the concentration of the measurement target gas 90 based on an intensity of the light 12 received by the first light receiving element 52. The first light receiving element 52 may be a photodiode, or may be a thermopile. The concentration measurement unit 50 may have a storage unit 54.

The gas sensor 100 may be a gas sensor of a so-called non-dispersive infrared (NDIR) type. The measurement target gas 90 absorbs an infrared light with a particular wavelength. The higher the concentration of the measurement target gas 90, the greater the absorption amount of light becomes. When the gas sensor 100 is of the NDIR type, the concentration measurement unit 50 measures the concentration of the measurement target gas 90 based on the intensity of the light 12 received by the first light receiving element 52.

The gas sensor 100 may be a gas sensor of a photoacoustic type. When the gas sensor 100 is a gas sensor of the photoacoustic type, the concentration measurement unit 50 may have a photoacoustic element that detects photoacoustic waves. When the light 12 is radiated to the measurement target gas 90, optical energy absorbed by a molecule of the measurement target gas 90 is converted into thermal energy. Thereby, the measurement target gas 90 expands. A pressure wave (photoacoustic wave) according to a change in the volume of the measurement target gas 90 is generated. The higher the concentration of the measurement target gas 90, the greater the amplitude of the pressure wave becomes. The photoacoustic element detects a change in the pressure of the measurement target gas 90 by measuring the pressure wave. The concentration measurement unit 50 measures the concentration of the measurement target gas 90 based on the change in the pressure detected by the photoacoustic element. The concentration measurement unit 50 may convert the change in the pressure detected by the photoacoustic element into the concentration of the measurement target gas 90.

FIG. 1B illustrates another example of the gas sensor 100 according to one embodiment of the present invention. The gas sensor 100 of the present example is different from the gas sensor 100 of FIG. 1A in that it further includes a second light receiving element 55 and a second optical filter 53. In the present example, the concentration measurement unit 50 has a second light receiving element 55 that receives the light 12 radiated to the measurement target gas 90. The second optical filter 53 may be provided between the light emitting element 10 and the second light receiving element 55 in the optical path of the light 12. The second optical filter 53 is a filter that transmits the light 12 in a wavelength band that is not absorbed by the measurement target gas 90. The second light receiving element 55 may receive the light 12 that was transmitted through the second optical filter 53.

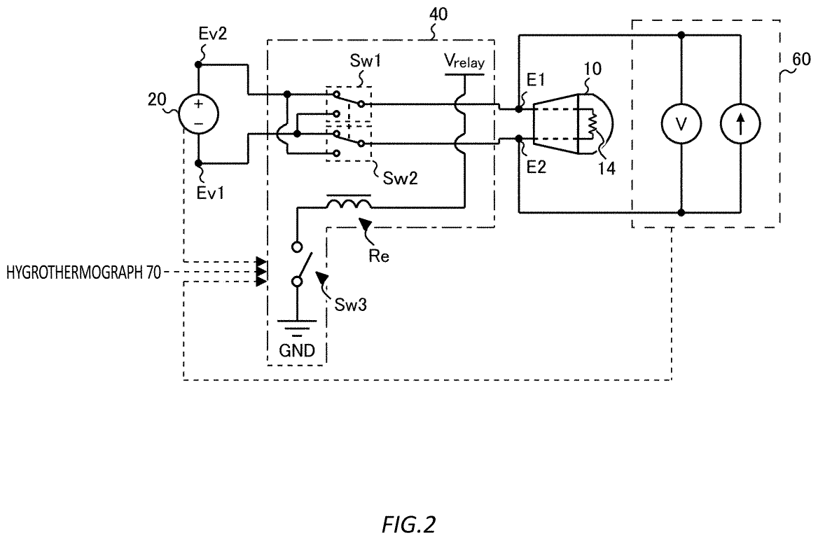

FIG. 2 is a circuit diagram illustrating an example of a connection relationship between a light emitting element 10 and a power supply unit 20 in the gas sensor 100 of FIG. 1A or FIG. 1B. The power supply unit 20 may be a constant-voltage power supply, or may be a constant-current power supply. In the example of FIG. 2, the power supply unit 20 is a constant-voltage power supply.

The light emitting element 10 may have a first terminal E1 and a second terminal E2. The first terminal E1 and the second terminal E2 are electric terminals. The light emitting element 10 emits light by the current that flows between the first terminal E1 and the second terminal E2. The light emitting element 10 of the present example has a filament 14 that is connected between the first terminal E1 and the second terminal E2.

The power supply unit 20 may have a first terminal Ev1 and a second terminal Ev2. The power supply unit 20 may maintain the first terminal Ev1 at a first potential V1, and may maintain the second terminal Ev2 at a second potential V2. The first potential V1 may be ground potential. The second potential V2 is a higher potential than the first potential V1. The power supply unit 20 supplies the direct current power to the light emitting element 10 through a potential difference between the first potential V1 and the second potential V2.

The gas sensor 100 includes a switching unit 40. In FIG. 2, the range of the switching unit 40 is indicated with a one-dot chain line. The switching unit 40 switches the polarity of the direct current power to be supplied to the first terminal E1 and the second terminal E2 of the light emitting element 10 based on a set condition. The switching unit 40 may switch the polarity of the voltage to be applied to the first terminal E1 and the second terminal E2. For example, the switching unit 40 switches said polarity by switching whether to relatively increase or decrease the potential of the second terminal E2 against the potential of the first terminal E1. The switching unit 40 of the present example switches the polarity of the electrical power to be supplied by the power supply unit 20 to the light emitting element 10 based on a set condition. The set condition is, for example, a period during which the electrical power of a first polarity is supplied or a period during which the electrical power of a second polarity is supplied, which are predetermined. The switching unit 40 switches an orientation of the current that flows through the filament 14 by switching the polarity of the electrical power. In the present specification, the polarity of the electrical power may refer to the orientation of the current that flows through the filament 14.

In the present example, the first polarity is a polarity where the current flows through the filament 14 in a direction from the second terminal E2 to the first terminal E1. That is, the first polarity is a state where the potential of the second terminal E2 is higher than the potential of the first terminal E1. In the example of FIG. 2, the first polarity is a state where the first terminal E1 of the light emitting element 10 and the first terminal Ev1 of the power supply unit 20 are connected, and the second terminal E2 and the second terminal Ev2 are connected. In the present example, the second polarity is a polarity where the current flows through the filament 14 in a direction from the first terminal E1 to the second terminal E2. That is, the second polarity is a state where the potential of the second terminal E2 is lower than the potential of the first terminal E1. In the example of FIG. 2, the second polarity is a state where the first terminal E1 and the second terminal Ev2 are connected, and the second terminal E2 and the first terminal Ev1 are connected. FIG. 2 is an example where the state of each switch is illustrated in a case of the second polarity.

In the example of FIG. 2, the switching unit 40 has a first switch Sw1 and a second switch Sw2. The first switch Sw1 selects either one of the first terminal Ev1 or the second terminal Ev2, and connects it to the first terminal E1 of the light emitting element 10. The second switch Sw2 selects the terminal, among the first terminal Ev1 and the second terminal Ev2, that is different from the first switch Sw1, and connects it to the second terminal E2 of the light emitting element 10. In FIG. 2, ranges of the first switch Sw1 and the second switch Sw2 are indicated with broken lines.

In the example of FIG. 2, the switching unit 40 has a third switch Sw3 and a relay element Re. In the present example, the switching unit 40 drives the first switch Sw1 and the second switch Sw2 via the relay element Re by turning the third switch Sw3 on or off. The switching unit 40 sets the polarity of the electrical power to be supplied to the power supply unit 20 to the first polarity by turning the third switch Sw3 on, and sets the polarity of said electrical power to the second polarity by turning the third switch Sw3 off.

The relay element Re may have a coil. The third switch Sw3 switches whether or not to cause the current to flow through said coil. The first switch Sw1 and the second switch Sw2 of the present example are switched to be on or off by a magnetic field generated by the current flowing through said coil.

The gas sensor 100 may further include a resistance acquisition unit 60. The resistance acquisition unit 60 acquires a resistance value R of the light emitting element 10. The resistance acquisition unit 60 may acquire the resistance value R of the filament 14. In FIG. 2, the range of the resistance acquisition unit 60 is indicated by a coarse dashed line. In the example of FIG. 2, the resistance acquisition unit 60 measures the voltage generated at both ends of the filament 14 by causing a constant current to flow between the first terminal E1 and the second terminal E2. The resistance acquisition unit 60 acquires the resistance value R from this voltage and the constant current that was caused to flow between the first terminal E1 and the second terminal E2.

The gas sensor 100 may further include a hygrothermograph 70. The hygrothermograph 70 will be described later.

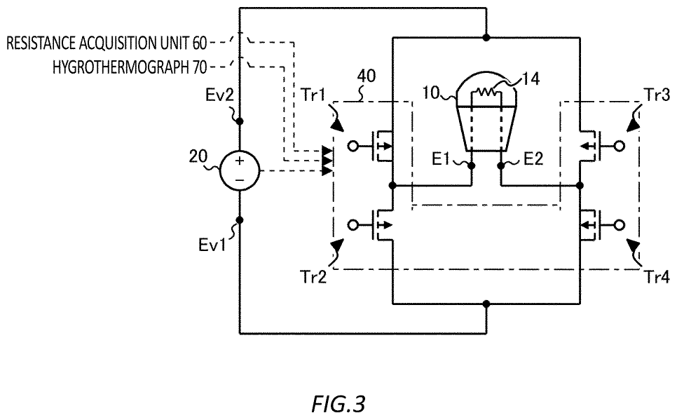

FIG. 3 is a circuit diagram illustrating another example of the connection relationship between the light emitting element 10 and the power supply unit 20 in the gas sensor 100 of FIG. 1A or FIG. 1B. In the present example, the power supply unit 20 is a constant-voltage power supply. In FIG. 3, the range of the switching unit 40 is indicated by a one-dot chain line. The gas sensor 100 of the present example has a resistance acquisition unit 60 that is similar to the example of FIG. 2.

In the example of FIG. 3, similarly to the example of FIG. 2, the first polarity is a state where the first terminal E1 of the light emitting element 10 and the first terminal Ev1 of the power supply unit 20 are connected, and the second terminal E2 and the second terminal Ev2 are connected. The second polarity is a state where the first terminal E1 and the second terminal Ev2 are connected and the second terminal E2 and the first terminal Ev1 are connected. FIG. 3 is an example illustrating the state of each transistor in a case of the second polarity.

In the present example, the switching unit 40 has a first transistor Tr1, a second transistor Tr2, a third transistor Tr3, and a fourth transistor Tr4. The first transistor Tr1 and the second transistor Tr2 in the present example switches the connection destination of the first terminal E1 of the light emitting element 10 between the first terminal Ev1 and the second terminal Ev2. Also, the third transistor Tr3 and the fourth transistor Tr4 switches the connection destination of the second terminal E2 of the light emitting element 10 between the first terminal Ev1 and the second terminal Ev2.

The first transistor Tr1 of the present example is arranged between the second terminal Ev2 and the first terminal Ev1. The second transistor Tr2 is arranged between the first transistor Tr1 and the first terminal Ev1. A connection node between the first transistor Tr1 and the second transistor Tr2 is connected to the first terminal E1 of the light emitting element 10. In addition, the third transistor Tr3 is arranged between the second terminal Ev2 and the first terminal Ev1. The fourth transistor Tr2 is arranged between the third transistor Tr3 and the first terminal Ev1. A connection node between the third transistor Tr3 and the fourth transistor Tr4 is connected to the second terminal E2 of the light emitting element 10.

In the present example, the switching unit 40 sets the polarity of the electrical power to the first polarity by turning the first transistor Tr1 and the fourth transistor Tr4 off and turning the second transistor Tr2 and the third transistor Tr3 on. The switching unit 40 sets the polarity of the electrical power to the second polarity by setting the first transistor Tr1 and the fourth transistor Tr4 on and turning the second transistor Tr2 and the third transistor Tr3 off.

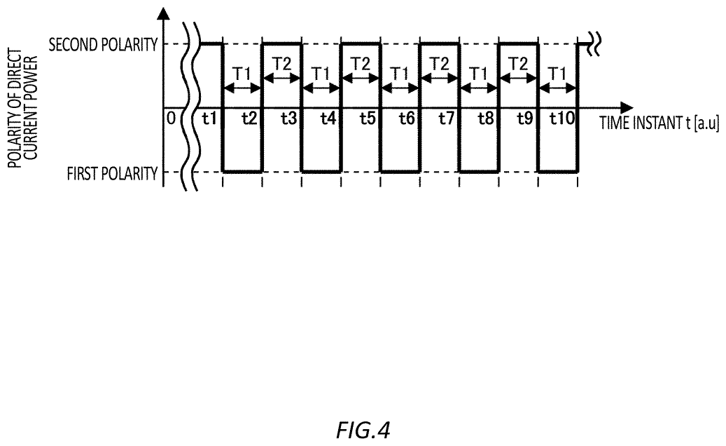

FIG. 4 illustrates an example of a relationship between the polarity of the direct current power and a time instant t. In the present example, the potential V at the first terminal E1 of the light emitting element 10 changes in accordance with the elapsed time. Note that, a potential with a phase that is different from that of the first terminal E1 by 180 degrees is applied to the second terminal E2 of the light emitting element 10.

The first terminal Ev1 and the second terminal Ev2 described in FIG. 2 and FIG. 3 are alternately connected to the first terminal E1 of the present example. The potential of the first terminal Ev1 is the first potential V1, and the potential of the second terminal Ev2 is the second potential V2. Therefore, the first potential V1 and the second potential V2 are alternately applied to the first terminal E1. During a period in which the first potential V1 is applied to the first terminal E1, the second potential V2 is applied to the second terminal E2. During a period in which the second potential V2 is applied to the first terminal E1, the first potential V1 is applied to the second terminal E2. The polarity of the direct current power applied to the light emitting element 10 is thereby alternately switched. The direct current power supplied by the power supply unit 20 may be pulsed. In the present example, a potential that changes in a pulsed manner is applied to the first terminal E1 and the second terminal E2. For example, in the example of FIG. 2, the power supply unit 20 (a constant-voltage power supply) supplies a pulsed electrical power by alternately supplying a constant voltage V_{const} and 0V. In the example of FIG. 2, the power supply unit 20 (a constant-voltage power supply) may supply a pulsed current by alternately supplying a constant voltage V_{const1} and a constant voltage V_{const2} with a different voltage from said V_{const1}.

The switching unit 40 (see FIG. 2 and FIG. 3) may switch the polarity of the direct current power according to set period T1 and period T2. In the example of FIG. 4, in the period T1, the first potential V1 is applied to the first terminal E1, and the second potential V2 is applied to the second terminal E2. In addition, in the period T2, the second potential V2 is applied to the first terminal E1, and the first potential V1 is applied to the second terminal E2. The periods T1 and T2 may have the same length, or may have different lengths from each other. The length of the period T1 may or may not change overtime. The length of the period T2 may or may not change overtime. The set period T1 and period T2 may be 0.1 second, may be 0.2 second, may be 1 second, may be 2 seconds, or may be 5 seconds.

The switching unit 40 may switch the polarity of the direct current power according to a set time instant t. In the example of FIG. 4, the polarity of the direct current power is switched at each time instant from time instants t1 to t6. The time instant t1 to the time instant t6 at which the polarity of the direct current power is switched may be predetermined. The time instant t1 to the time instant t6 may be on different dates from one another.

A part of the filament 14 of the light emitting element 10 may evaporate during activation of the light emitting element 10. When a part of the filament 14 evaporates, the filament 14 becomes thin. The filament 14 thereby tends to break. When a direct current power with the same polarity continues to be supplied to the light emitting element 10, the same place of the filament 14 tends to continue evaporating. Thus, the filament 14 tends to be locally thin. At the gas sensor 100, the polarity of the direct current power is switched by the switching unit 40 based on a set condition. For example, the switching unit 40 switches the polarity of the direct current power periodically. The place of evaporation in the filament 14 thereby tends to be distributed. The lifetime of the filament 14 may thereby be increased.

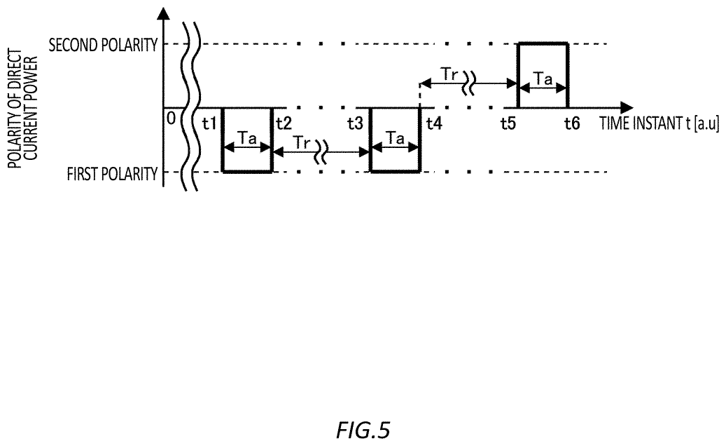

FIG. 5 illustrates another example of the relationship between the polarity of the direct current power and the time instant t. In the present example, the power supply unit 20 supplies the direct current power with the first polarity to the light emitting element 10 from the time instant t1 to the time instant t2 and from the time instant t3 to the time instant t4, and supplies the direct current power with the second polarity to the light emitting element 10 from the time instant t5 to the time instant t6. In the present example, the power supply unit 20 does not supply the direct current power to the light emitting element 10 from the time instant t2 to the time instant t3 and from the time instant t4 to the time instant t5.

The period from the time instant t1 to the time instant t2, the period from the time instant t3 to the time instant t4, and the period from the time instant t5 to the time instant t6 is defined as a period Ta. The period from the time instant t2 to the time instant t3 and the period from the time instant t4 to the time instant t5 is defined as period Tr. The period Tr may be equal to the period Ta, may be ten times or more of the period Ta, may be twenty times or more, or may be a hundred times or more.

The switching unit 40 may switch the polarity of the direct current power based on a number of times of activation of the power supply unit 20. For example, the switching unit 40 switches the polarity of the direct current power, when the number of times of activation of the power supply unit 20 exceeds a predetermined number of times. Said predetermined number of times may be twice, may be five times, or may be ten times. Activation of the power supply unit 20 refers to transition from a state where the power supply unit 20 is not activated to a state where the power supply unit 20 is activated. The state where the power supply unit 20 is activated is the state of the power supply unit 20 during the period Ta in the example of FIG. 5. The state where the power supply unit 20 is not activated is the state of the power supply unit 20 during the period Tr in the example of FIG. 5. In the example of FIG. 5, the switching unit 40 switches the polarity of the direct current power to the second polarity when the number of times of activation of the direct current power with the first polarity exceeds two times.

The switching unit 40 may switch the polarity of the direct current power based on the number of times of activation of the light emitting element 10. For example, the switching unit 40 switches the polarity of the direct current power when the number of times of activation of the light emitting element 10 exceeds a predetermined number of times. Said predetermined number of times may be twice, may be five times, or may be ten times. Activation of the light emitting element 10 refers to transition from a state where the direct current power is not supplied to the light emitting element 10 to a state where the direct current output is supplied thereto. The state where the direct current power is not supplied to the light emitting element 10 refers to a state where a current does not flow through the light emitting element 10. The activation of the light emitting element 10 refers to transition from a state where the current does not flow through the light emitting element 10 to a state where the current flows through the light emitting element 10 by being supplied with a direct current output. The state where the direct current power is not supplied to the light emitting element 10 is the state of the direct current power during the period Tr in the example of FIG. 5. In the example of FIG. 5, the switching unit 40 switches the polarity of the direct current power to the second polarity when the number of times of activation of the light emitting element 10 with the first polarity exceeds two times.

As described above, the period Tr may be equal to the period Ta, may be ten times or more of the period Ta, may be twenty times or more, or may be a hundred times or more. For example, when the period Ta is one second and the period Tr is less than ten seconds, the direct current power may be considered to be continuously supplied to the light emitting element 10. That is, the supply of the direct current power at the time instant t3 and the time instant t5 may not be included in the number of times of activation of the light emitting element 10. For example, when the period Ta is one second and the period Tr is ten seconds or more, the supply of the direct current power at the time instant t3 and the time instant t5 may be included in the number of times of activation of the light emitting element 10.

In the period Tr during which the direct current power is not supplied, the filament 14 may be at a low temperature. When the temperature of the filament 14 is low, the filament may have a low resistance value R. When the power supply unit 20 is a constant-voltage power supply, at the moment the direct current power is supplied (that is, the moment period Ta begins), a large direct current may flow through the filament 14. When there is a place where the filament 14 is thin, said place may rapidly generate heat due to the large direct current. When the power supply unit 20 is a constant-current power supply, regardless of the temperature of the filament 14, the moment the direct current power is supplied (that is, the moment the period Ta begins), a constant direct current flows through the filament 14. When there is a place where the filament 14 is thin, said place may rapidly generate heat due to this constant direct current. Thus, in either case of the power supply unit 20 being a constant-voltage power supply or a constant-current power supply, the greater the number of times of activation of the light emitting element 10, the more the evaporation amount at the same place, which is a part of the filament 14, tends to become. By switching the polarity of the direct current power by the switching unit 40 based on the number of times of activation of the light emitting element 10, the part in the filament 14 that evaporates tends to be distributed.

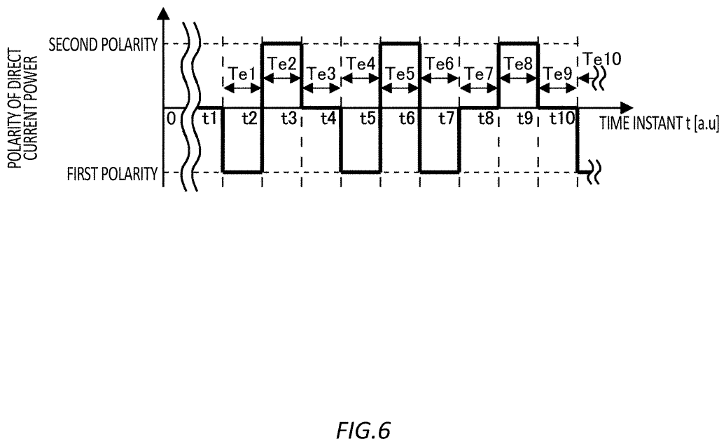

FIG. 6 illustrates another example of the relationship between the polarity of the direct current power and the time instant t. In the present example, the power supply unit 20 starts supplying the direct current power with the first polarity at the time instant t1, the time instant t4, the time instant t6, and the time instant t10, and starts supplying the direct current power with the second polarity at the time instant t2, the time instant t5, and the time instant t8. In the present example, the power supply unit 20 continues supplying the direct current power with the first polarity during the period Te1, the period Te4, the period Te6, and the period Te10, and continues supplying the direct current power with the second polarity during the period Te2, the period Te5, and the period Te8. In the present example, the power supply unit 20 does not supply the direct current power during the period Te3, the period Te7, and the period Te9. The lengths of the period Te1 to the period Te10 may be equal, or may be different from one another.

The power supply unit 20 may acquire the supply time for which the direct current power with the first polarity is supplied. For example, when the current time instant t is any time instant in the period Te7, the power supply unit 20 acquires, as the supply time, the total time of the period Te1, the period Te4, and the period Te6. The supply time for which the power supply unit 20 supplies the direct current power with the first polarity may be preset. Said supply time may be preset by a date or a time instant.

The switching unit 40 may switch the polarity of the direct current power to the second polarity based on the supply time. For example, the switching unit 40 switches the polarity of the direct current power to the second polarity when the supply time exceeds a predetermined time. As described above, when the direct current power with the same polarity continues to be supplied to the light emitting element 10, W (tungsten) at the same place of the filament 14 tends to continue to evaporate. Thereby, the filament 14 tends to become locally thin. By switching the polarity of the direct current power by the switching unit 40 based on the supply time, a part in the filament 14 where W (tungsten) evaporates tends to be distributed. The lifetime of the filament 14 may thereby be increased.

The switching unit 40 may switch the polarity of the direct current power based on the resistance value R of the light emitting element 10. For example, the switching unit 40 may switch the polarity when the resistance value R is increased by a predetermined percentage. The resistance value R of the light emitting element 10 at a first time instant is defined as R0, and the resistance value R of the light emitting element 10 at a second time instant that is later than the first time instant is defined as R1. The percentage of increase of the resistance value R may be defined by (R1/R0). The predetermined percentage of increase may be 0.05%, may be 0.1%, may be 0.2%, may be 0.5%, or may be 1%.

When the activation of the light emitting element 10 is started at the first time instant, the resistance value R0 is a resistance value R before the current flows through the filament 14. When the time instant t1 in FIG. 6 is the first time instant, the second time instant is the time instant t2, for example. The resistance value R at the time instant t1 is R0, and the resistance value R at the time instant t2 is R1. In the example of FIG. 6, the switching unit 40 switches the polarity from the first polarity to the second polarity when (R1/R0) exceeds the predetermined percentage at the time instant t2. When the time instant t2 in FIG. 6 is the first time instant, the second time instant is the time instant t3, for example. The resistance value R at the time instant t2 is R0, and the resistance value R at the time instant t3 is R1. In the example of FIG. 6, the switching unit 40 switches the polarity from the second polarity to the first polarity when (R1/R0) exceeds the predetermined percentage at the time instant t3. In this manner, the switching unit 40 may switch the polarity every time the resistance value R is increased by the predetermined percentage.

When W (tungsten) generates heat in the filament 14, the resistance value R may be increased. When the resistance value R is increased, the filament 14 tends to be further heated. The evaporation amount of W (tungsten) thereby tends to be increased. The filament 14 thereby tends to break. By switching the polarity by the switching unit 40 based on the resistance value R, the place in the filament 14 where W (tungsten) evaporates tends to become uniform. The lifetime of the filament 14 may thereby be increased.

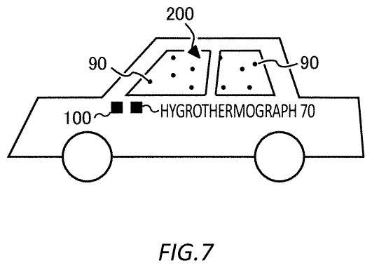

FIG. 7 illustrates an example of a measurement target 200. The measurement target 200 of the present example is an indoor space of a vehicle. The measurement target 200 includes a measurement target gas 90. In the present example, the measurement target gas 90 is included in the indoor space of the vehicle. A hygrothermograph 70 measures the temperature or humidity of the measurement target 200. In the present example, the hygrothermograph 70 measures the temperature or humidity of the indoor space.

In the present example, the gas sensor 100 is provided in the measurement target 200. The gas sensor 100 may or may not include the hygrothermograph 70. In a case where the hygrothermograph 70 is provided in the vehicle, the gas sensor 100 may not include the hygrothermograph 70. The hygrothermograph 70 provided in the vehicle may send the measured temperature or humidity to the gas sensor 100.

The switching unit 40 may switch the polarity of the direct current power based on the temperature or humidity of the measurement target 200. For example, the switching unit 40 reduces the the cycle of switching the polarity when the temperature or humidity of the measurement target 200 is higher, and increases the cycle of switching the polarity when the temperature or humidity of the measurement target 200 is lower. When the measurement target 200 is the indoor space of the vehicle, the temperature of the measurement target 200 tends to become significantly higher than the temperature outside the measurement target 200. In a case of the indoor space of the vehicle, the temperature of the measurement target 200 may become about 60 to 70° C. In such a case, the temperature of the filament 14 during activation of the light emitting element 10 tends to become higher. The resistance value R of the filament 14 thereby tends to be further increased. The filament 14 thereby tends to be further heated.

The higher the humidity of the measurement target 200, the higher the temperature of the filament 14 during activation of the light emitting element 10 tends to become. The resistance value R of the filament 14 thereby tends to be further increased. The filament 14 thereby tends to be further heated.

By the switching unit 40 reducing the cycle of switching the polarity when the temperature or humidity of the measurement target 200 is higher, a part in the filament 14 where W (tungsten) is evaporated tends to be distributed before the part of the filament 14 becomes locally thin. The lifetime of the filament 14 may thereby be increased.

When the measurement target 200 is the indoor space of the vehicle, the switching unit 40 may switch the polarity based on at least one of a number of times of activation of an engine of the vehicle, travel distance, location information, or a remaining amount of fuel. The power supply unit 20 (see FIG. 1A) in the vehicle is activated in accordance with a key switch being turned ON. The engine of the vehicle is thereby activated. Therefore, the number of times of activation of the engine is basically equal to the number of times of the key switch being turned ON. In a case where the vehicle is of a push start type by a smart key, the engine of the vehicle is activated in accordance with a push start. Therefore, the number of times of activation of the engine is equal to the number of times of the push start. In a case where the number of times of the key switch being turned ON or the number of times of the push start is defined as m (m is a natural number), when the power supply unit 20 supplies the direct current power with the first polarity for the 2m-1^{th} time, the switching unit 40 may switch the polarity of the direct current power to be supplied for the 2m^{th} time to the second polarity. In addition, even when the key switch is rotated halfway (ACC state) to activate the power supply unit 20 but not the engine of the vehicle, it may be included in the number of times of the key switch being turned ON or the number of times of the push start. With respect to a vehicle that is driven by a motor such as an electric vehicle or a fuel-cell electric vehicle, the number of times of the motor being driven may be used instead of the number of times of activation of the engine, and even when the power supply unit 20 is activated but the motor of the vehicle is not activated, it may be included in the number of times of the key switch being turned ON or the number of times of the push start.

The power supply unit 20 may be a battery mounted on the vehicle, such as a lead storage battery or a lithium ion battery, for example. When the battery is mounted on the gas sensor 100, the power supply unit 20 may be a battery installed in the gas sensor 100, for example, a primary battery such as an alkaline battery or a manganese battery, or a secondary battery such as a lithium ion battery or a nickel hydrogen battery.

The switching unit 40 may switch the polarity of the direct current power based on an activation time of the engine. The activation time of the engine refers to a period from the time instant at which the engine is activated by the user of the vehicle to the current time instant. When the gas sensor 100 is activated during activation of the engine, the longer the activation time of the engine, the longer the activation time of the gas sensor 100 becomes. Thus, when the polarity of the direct current power is not switched at the activation time of the gas sensor 100, a part of the filament 14 tends to become locally thin. Thus, when the activation time of the engine exceeds a predetermined time, the switching unit 40 may switch the polarity of the direct current power.

The travel distance of the vehicle may be a travel distance from when the engine is activated by the user of the vehicle and the user starts to travel on said vehicle to the current time instant. Said travel distance may be acquired based on location information of the vehicle at the time instant when the engine was activated by the user and location information of the vehicle at the current time instant. Said travel distance may be a distance between a position of the vehicle at the time instant when the engine was activated by the user and a position of the vehicle at the current time instant. The longer the travel distance, the longer the activation time of the engine may become. Thus, the when the travel distance of the vehicle exceeds a predetermined distance, the switching unit 40 may switch the polarity of the direct current power.

The remaining amount of the fuel is an amount obtained by deducting, from a predetermined amount of fuel (for example, the amount of fuel when the fuel is filled up), a consumption amount of fuel due to the vehicle travelling. For the remaining amount of fuel, a value sensed for the remaining amount of fuel at the current time instant by a sensor or the like may be used. The longer the travel distance of the vehicle, the more the consumption amount of fuel may become. Thus, the switching unit 40 may switch the polarity of the direct current power based on the remaining amount of fuel. The switching unit 40 may switch the polarity of the direct current power when the remaining amount of fuel is less than a predetermined remaining amount.

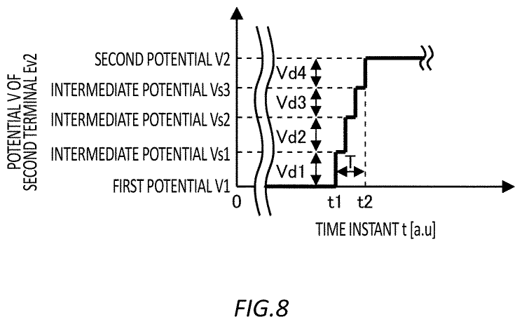

FIG. 8 illustrates an example of a relationship between a potential V of the second terminal Ev2 and a time instant t. When the power supply unit 20 is a constant-voltage power supply, said constant-voltage power supply may increase the potential difference between a first potential V1 and a second potential V2 in a stepwise manner. The constant-voltage power supply may increase the potential V of the second terminal Ev2 from the first potential V1 to the second potential V2 in a stepwise manner, while maintaining the first terminal Ev1 at the first potential V1.

The constant-voltage power supply may increase the potential V of the second terminal Ev2 from the first potential V1 to an intermediate potential Vs1, while maintaining the first terminal Ev1 at the first potential Vs1. The constant-voltage power supply may increase the potential V of the second terminal Ev2 from the intermediate potential Vs1 to a intermediate potential Vs2, after a predetermined time has elapsed since the potential V of the second terminal Ev2 is increased to the intermediate potential Vs1. When the potential V of the second terminal Ev2 is increased from the first potential V1 to the intermediate potential Vs1, the current I that flows through the filament 14 changes. Since the current I may transiently change, the current I may not have completely changed to a current I corresponding to the intermediate potential Vs1 immediately after the increase to the intermediate potential Vs1. The predetermined time to elapse after the potential V of the second terminal Ev2 is increased to the intermediate potential Vs1 refers to the time it takes for the change of the current I that flows through the filament 14 to complete and for the change to the current I corresponding to the intermediate potential Vs1 to end. Similarly, the constant-voltage power supply may increase the potential V of the second terminal Ev2 to the second potential V2.

The number of the intermediate potentials Vs may be at least one. In the present example, there are three intermediate potentials Vs. The number of the intermediate potentials Vs may be two, or may be four or more. The potential difference between adjacent potentials is defined as a potential difference Vd. When there are n intermediate potentials Vs (n is an integral of two or more), there are n+1 potential differences Vd from a potential difference Vd₁ between the first potential V1 and the intermediate potential Vs1 to a potential difference Vdₙ₊₁ between the intermediate potential Vsn and the second potential V2. The n+1 potential differences Vd may be the same as one another, or may be different from one another.

Among the n+1 potential differences Vd, the potential difference Vd₁ may be the smallest, and the potential difference Vdₙ₊₁ may be the greatest. The current that initially flows through the filament 14 is thereby reduced. Thus, the filament 14 is made to hardly break. The lifetime of the filament 14 may thereby be increased. In the example of FIG. 8, for example, the potential difference Vd1 is set to be smaller than the potential difference Vd2, the potential difference Vd2 is set to be smaller than the potential difference Vd3, and the potential difference Vd3 is set to be smaller than the potential difference Vd4.

Among the n+1 potential differences Vd, the potential difference Vd₁ may be the greatest, and the potential difference Vdₙ₊₁ may be the smallest. The current that initially flows through the filament 14 is thereby increased. Thus, the second terminal Ev2 tends to reach the second potential V2 quickly. In the example of FIG. 8, for example, the potential difference Vd1 is set to be greater than the potential difference Vd2, the potential difference Vd2 is set to be greater than the potential difference Vd3, and the potential difference Vd3 is set to be greater than the potential difference Vd4.

In the present example, the time instant t1 is the time instant at which the constant-voltage power supply increases the potential V of the second terminal Ev2 from the first potential V1 to the intermediate potential Vs, and the the time instant t2 is the time instant at which the constant-voltage power supply increases the potential V of the second terminal Ev2 from the intermediate potential Vs to the second potential V2. The period T is a period between the time instant t1 and the time instant t2. The constant-voltage power supply may cause the potential V of the second terminal Ev2 to change in a stepwise manner during the period T from the first potential V1 to the second potential V2.

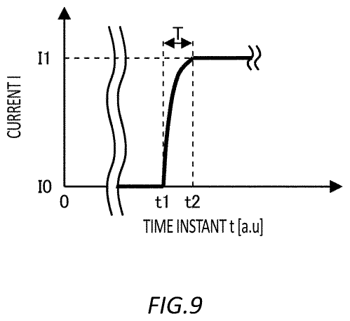

FIG. 9 illustrates an example of a relationship between a current I that flows through the filament 14 and a time instant t. FIG. 9 is an example of the relationship between the current I and a time instant t, in a case where the potential V of the second terminal Ev2 changes as illustrated in FIG. 8 in accordance with the change in the time instant t. Due to the potential V of the second terminal Ev2 being increased in a stepwise manner, the current I tends to gradually increase during the period T. In the present example, the power supply unit 20 is a constant-voltage power supply. Thus, during the elapse of the predetermined time after the potential V of the second terminal Ev2 is increased to the intermediate potential Vs1, the current I fall according to the rise in the resistance value R of the filament 14. Thus, the speed of the filament 14 becoming thin is reduced.

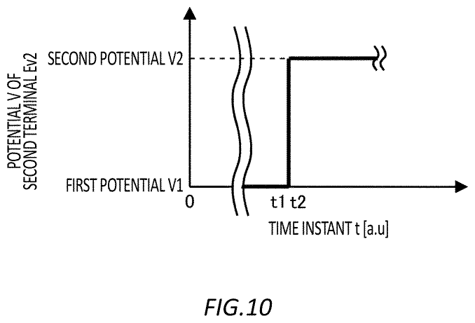

FIG. 10 illustrates a comparative example of a relationship between the potential V of the second terminal Ev2 and the time instant t. In the present comparative example, the constant-voltage power supply does not cause the potential difference between the first potential V1 and the second potential V2 to change in a stepwise manner. The period T in the present comparative example is negligible compared to the period T in the example of FIG. 8.

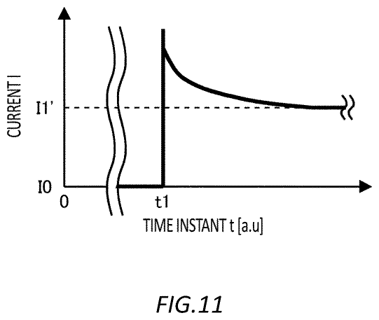

FIG. 11 illustrates a comparative example of a relationship between the current I that flows through the filament 14 and the time instant t. FIG. 11 is an example of the relationship between the current I and a time instant t, in a case where the potential V of the second terminal Ev2 changes such as illustrated in FIG. 10 in accordance with the change in the time instant t. In the present comparative example, since the constant-voltage power supply does not cause the potential difference between the first potential V1 and the second potential V2 to change in a stepwise manner, the current I tends to spike at the time instant t1. After spiking at the time instant t1, the current I falls to a constant current I1′ according to the rise in the resistance value R of the filament 14. In the comparative example, since the current I tends to spike, the a temperature rise tends to occur locally in the filament 14. Thus, the place where the filament 14 is starting to become thin becomes easier to break. Thus, the lifetime of the filament 14 tends to be decreased.

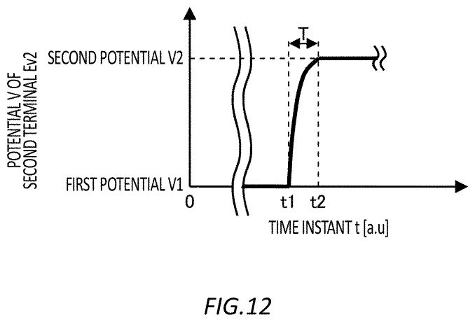

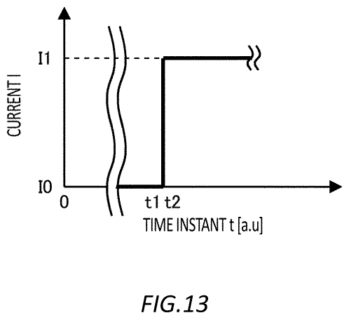

FIG. 12 illustrates another example of the relationship between the potential V of the second terminal Ev2 and the time instant t. FIG. 13 illustrates an example of the relationship of the current I that flows through the filament 14 and a time instant t. FIG. 12 and FIG. 13 are examples of a case where the power supply unit 20 is a constant-current power supply. When the power supply unit 20 is a constant-current power supply, the current I that flows through the filament 14 is constant. Thus, similar to the case of the constant-voltage power supply, the current that rapidly flows through the filament 14 in accordance with the start of supply of the direct current power is made to hardly generated. Therefore, a rapid heat generation of the filament 14 due to the current rapidly flowing through the filament 14 is made to hardly occur. Thus, evaporation of the filament 14 due to this heat generation may be suppressed. Thus, the filament 14 is made to hardly break. Thus, the lifetime of the filament 14 tends to be increased.

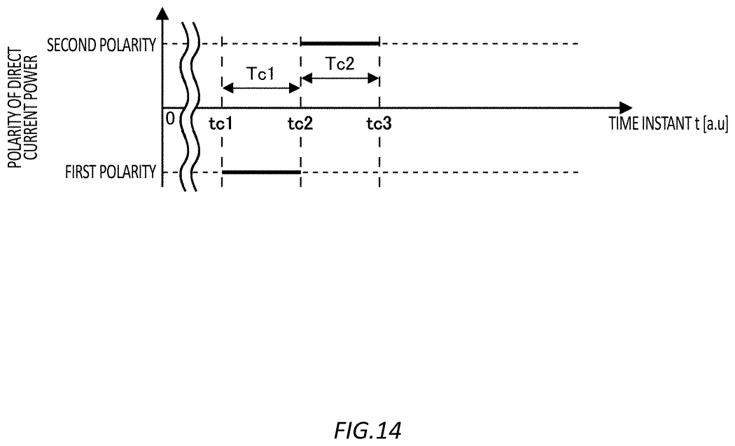

FIG. 14 illustrates another example of the relationship between the polarity of the direct current power and the time instant t. In the present example, the power supply unit 20 starts supplying the direct current power with the first polarity at a time instant tc1, and starts supplying the direct current power with the second polarity at a time instant tc2. In the present example, the power supply unit 20 supplies the direct current power with the first polarity during the first period Tc1, and supplies the direct current power with the second polarity during the second period Tc2. In the present example, the concentration measurement unit 50 (see FIG. 1A) measures the first concentration of the measurement target gas 90 during the first period Tc1, and measures the second concentration of the measurement target gas 90 during the second period Tc2.

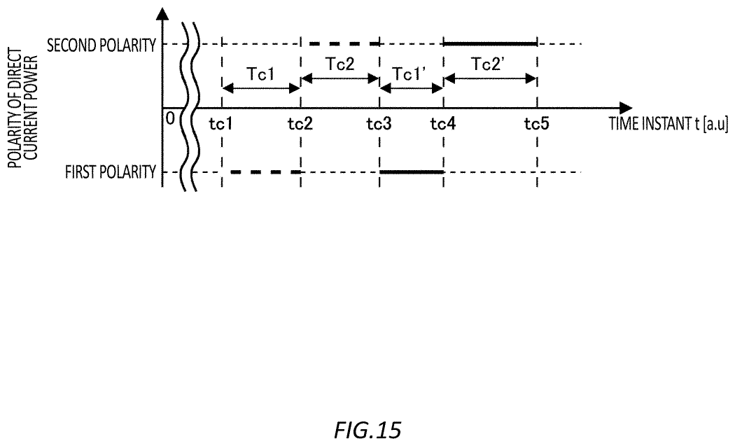

FIG. 15 illustrates another example of the relationship between the polarity of the direct current power and the time instant t. The concentration measurement unit 50 (see FIG. 1A) may correct at least one of the first period Tc1 or the second period Tc2 based on the first concentration and the second concentration. For example, the concentration measurement unit 50 corrects the first period Tc1 to be shorter and corrects the second period Tc2 to be longer, based on the first concentration and the second concentration. In FIG. 15, the first period Tc1 is a first period Tc1 after the correction, and the second period Tc 2 is a second period Tc2 after the correction. In the example of FIG. 15, the first period Tc1 is a period from a time instant tc3 to a time instant tc4, and the second period Tc2 is a period from the time instant tc4 to a time instant tc5. In FIG. 15, the first period Tc1 and the second period Tc2 in FIG. 14 are indicated by coarse dashed lines.

The concentration measurement result of the measurement target gas 90 by the concentration measurement unit 50 is ideally the same, regardless of the polarity of the direct current power. However, the resistance value R of the filament 14 in the case of respective polarities may be different from one another according to the usage time of the gas sensor 100 in the case of respective polarities. In such a case, the current I that flows through the filament 14 may be different from one another in the case of respective polarities. The light emission amount of the light emitting element 10 in the case of respective polarities or the light emittance intensity distribution at the places of the filament where the light is emitted may thereby be different from one another. The concentration measurement result in the case of respective polarities may thereby be different from one another.

In the example of FIG. 15, when the first concentration measured in the first period Tc1 is lower smaller than than the second concentration measured in the second period Tc2, the likelihood is high that the light emission amount of the light emitting element 10 in the case of the first polarity is smaller than than the light emission amount of the light emitting element 10 in the case of the second polarity. Thus, the likelihood is high that the resistance value R of the filament 14 in the case of the first polarity is higher than the resistance value R of the filament 14 in the case of the second polarity. Thus, the likelihood is high that the lifetime of the filament 14 is shorter in the case of the first polarity than in the case of the second polarity. In such a case, the concentration measurement unit 50 may set the first period Tc1′ to be shorter than the first period Tc1 by correcting the first period Tc1. The concentration measurement unit 50 may set the second period Tc2′ to be longer than the second period Tc2 by correcting the second period Tc2. Similarly, when the first concentration measured in the first period Tc1 is greater than the second concentration measured in the second period Tc2, the concentration measurement unit 50 may set the first period Tc1′ to be longer than the first period Tc1 by correcting the first period Tc1, and may set the second period Tc2′ to be shorter than the second period Tc2 by correcting the second period Tc2.

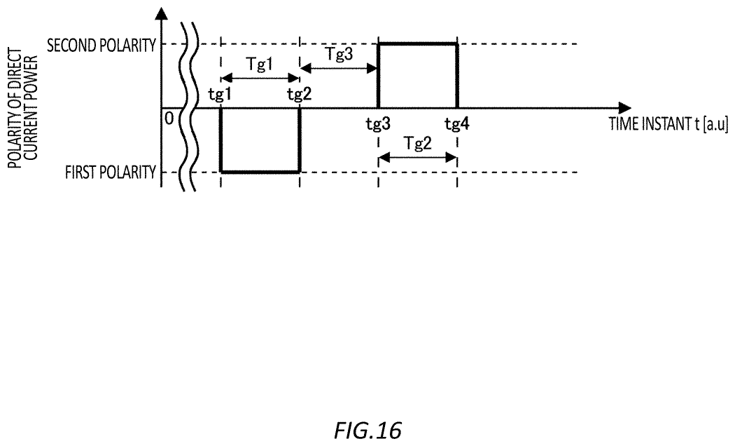

FIG. 16 illustrates another example of the relationship between the polarity of the direct current power and the time instant t. In the present example, the period from a time instant tg1 to a time instant tg2 is defined as a first period Tg1, the period from the time instant tg2 to a time instant tg3 is defined as a third period Tg3, and the period from the time instant tg3 to a time instant tg4 is defined as a second period Tg2. In the present example, during the first period Tg1 and the second period Tg2, the first terminal Ev1 (see FIG. 2 and FIG. 3) is maintained at the first potential V1, and the second terminal Ev2 (see FIG. 2 and FIG. 3) is maintained at the second potential V2. That is, in the present example, during the first period Tg1 and the second period Tg2, the power supply unit 20 supplies the direct current power of either the first polarity or the second polarity. In the example of FIG. 16, the power supply unit 20 supplies the direct current power with the first polarity in the first period Tg1, and supplies the direct current power with the second polarity in the second period Tg2.

In the third period Tg3, the first terminal Ev1 (see FIG. 2 and FIG. 3) may be maintained at the first potential V1, and the second terminal Ev2 (see FIG. 2 and FIG. 3) may be maintained at the first potential V1 or more and less than the second potential V2. In the third period Tg3, the power supply unit 20 may supply the direct current power of when the second terminal Ev2 is maintained at the first potential V1 or more and less than the second potential V2. In the example of FIG. 16, the first terminal Ev1 and the second terminal Ev2 are maintained at the first potential V1 and said first potential V1 is 0V. Thus, in the example of FIG. 16, the power supply unit 20 does not supply the direct current power in the third period Tg3.

The concentration measurement unit 50 may measure the concentration of the measurement target gas 90 based on a first intensity of the light 12 received by the first light receiving element 52 in the first period Tg1 and a third intensity of the light 12 received by the first light receiving element 52 in the third period Tg3. For example, the concentration measurement unit 50 calculates the concentration of the measurement target gas 90 based on the difference (the first intensity—the third intensity) between the first intensity of the light 12 measured in the first period Tg1 and the third intensity of the light 12 measured in the third period Tg3. The concentration measurement unit 50 may calculate the concentration of the measurement target gas 90 based on the ratio (the first intensity/the third intensity) of the first intensity of the light 12 measured in the first period Tg1 and the third intensity of the light 12 measured in the third period Tg3.

In the example of FIG. 16, the power supply unit 20 does not supply the direct current power in the third period Tg3. Thus, the concentration of the measurement target gas 90 in the third period Tg3 may be zero. However, the first light receiving element 52 may measure the third intensity as a value other than zero by receiving light other than the light 12 (see FIG. 1A) emitted by the light emitting element 10. In such a case, even when the direct current power is not supplied in the third period Tg3, the concentration measurement unit 50 may measure the concentration of the measurement target gas 90 as a value other than zero. Thus, the concentration measurement unit 50 may measure an accurate concentration of the measurement target gas 90 by measuring the concentration of the measurement target gas 90 based on the first intensity of the light 12 received by the first light receiving element 52 in the first period Tg1 and the third intensity of the light 12 received by the first light receiving element 52 in the third period Tg3.

The power supply unit 20 may supply, in the third period Tg3, the direct current power of when the second terminal Ev2 is maintained to be greater than the first potential V1 and less than the second potential V2. In this case, the power supply unit 20 supplies the direct current power that is smaller than the direct current power of when the first terminal Ev1 is maintained at the first potential V1 and the second terminal Ev2 is maintained at the second potential V2. The third intensity may be the intensity of the light 12 received by the first light receiving element 52 when the power supply unit 20 supplies such a direct current power.

The concentration measurement unit 50 may measure the concentration of the measurement target gas 90 based on the second intensity of the light 12 received by the first light receiving element 52 in the second period Tg2 and the third intensity of the light 12 received by the first light receiving element 52 in the third period Tg3. For example, the concentration measurement unit 50 calculates the concentration of the measurement target gas 90 based on a difference (the second intensity—the third intensity) between the second intensity of the light 12 measured in the second period Tg2 and the third intensity of the light 12 measured in the third period Tg3. The concentration measurement unit 50 may calculate the concentration of the measurement target gas 90 based on a ratio (the second intensity/the third intensity) of the second intensity of the light 12 measured in the second period Tg2 and the third intensity of the light 12 measured in the third period Tg3.

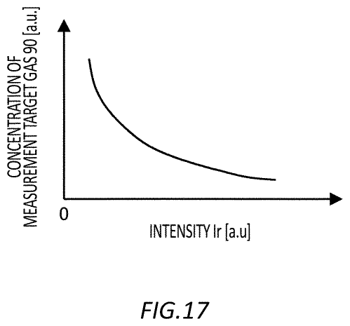

FIG. 17 illustrates an example of a relationship between the intensity Ir of the light 12 and the concentration of the measurement target gas 90. The relationship between the intensity Ir of the light 12 and the concentration of the measurement target gas 90 is the relationship illustrated in FIG. 17, for example. The higher the concentration of the measurement target gas 90, the more the amount of light absorbed in the gas molecule may become. Thus, the intensity of the light 12 received by the first light receiving element 52 (see FIG. 1A) may tend to be reduced. The lower the concentration of the measurement target gas 90, the less the amount of light absorbed in the gas molecule may become. Thus, the intensity of the light 12 received by the first light receiving element 52 tends to increase. The relationship between the intensity Ir of the light 12 and the concentration of the measurement target gas 90 may be measured in advance. The relationship between the intensity Ir of the light 12 measured in advance and the concentration of the measurement target gas 90 may be stored in the storage unit 54 (see FIG. 1A).

The intensity Ir of the light 12 may be a difference between the first intensity and the third intensity of the light 12, or may be a ratio of the first intensity and the third intensity of the light 12. The intensity Ir of the light 12 may depend on the temperature of the inner space 32 (see FIG. 1A). Thus, the difference or ratio between the first intensity and the third intensity may be the difference or ratio after correcting for the temperature of the inner space 32. The intensity Ir of the light 12 may be a difference between the second intensity and the third intensity of the light 12, or may be a ratio of the second intensity and the third intensity of the light 12. The difference or ratio between the second intensity and the third intensity may be the difference or ratio after correcting for the temperature of the inner space 32.

The concentration measurement unit 50 may measure the concentration of the measurement target gas 90 based on the difference or ratio between the first intensity in the first period Tg1 and the third intensity in the third period Tg3 and the relationship between the intensity Ir of the light 12 and the concentration of the measurement target gas 90 that is stored in the storage unit 54. The concentration measurement unit 50 may measure the concentration of the measurement target gas 90 based on the difference or ratio between the second intensity in the second period Tg2 and the third intensity in the third period Tg3 and the relationship between the intensity Ir of the light 12 and the concentration of the measurement target gas 90 that is stored in the storage unit 54.

The second light receiving element 55 (see FIG. 1B) may be a reference element. The second light receiving element 55 receives the light 12 that transmitted through the second optical filter 53. The second optical filter 53 is a filter that transmits the light 12 in a wavelength band that is not absorbed by the measurement target gas 90. The first light receiving element 52 receives the light 12 that transmitted through the first optical filter 51. The first optical filter 51 is a filter that transmits the light 12 in a wavelength band which is absorbed by the measurement target gas 90. The concentration measurement unit 50 may measure the concentration of the measurement target gas 90 based on a difference between the amount of the light received by the first light receiving element 52 and the amount of the light received by the second light receiving element 55. The concentration measurement unit 50 may thereby accurately measure the concentration of the measurement target gas 90.

The second light receiving element 55 (see FIG. 1B) may be an element of which the light receiving characteristic has been calibrated. The light receiving characteristic of the first light receiving element 52 may change overtime. The change overtime is a deterioration overtime, for example. The concentration measurement unit 50 may calibrate the light receiving characteristic of the first light receiving element 52 with the light receiving characteristic of the second light receiving element 55 and measure the concentration of the measurement target gas 90 based on the calibrated light receiving characteristic. The concentration measurement unit 50 may thereby measure an accurate concentration, even when the light receiving characteristic of the first light receiving element 52 has changed overtime.

The second light receiving element 55 may detect a gas that is different from that detected by the first light receiving element 52. The first light receiving element 52 may detect the measurement target gas 90, and the second light receiving element 55 may detect a reference gas. The reference gas may be used to estimate a dilution rate of the measurement target gas 90. For example, the first light receiving element 52 may detect light in a wavelength band corresponding to alcohol, and the second light receiving element 55 may detect light in a wavelength band corresponding to carbon dioxide. The concentration of carbon dioxide included in human breath is generally constant. Thus, the degree of dilution of the breath that is measured by the gas sensor 100 can be estimated by detecting, by the gas sensor 100, the concentration of carbon dioxide. Since it can be considered that the dilution rate of alcohol included in the breath is equivalent of the dilution rate of carbon dioxide included in the breath, the concentration of alcohol included in the breath can be estimated based on the concentration of alcohol detected by the gas sensor 100 and the estimated dilution rate. Besides, various combinations of the types of gas to be detected by each light receiving element is conceivable, such as detecting light that corresponds to carbon dioxide by the first light receiving element 52 and detecting light that corresponds to carbon monoxide by the second light receiving element 55.

The light emission characteristic of the light emitting element 10 may change overtime. The change overtime is a deterioration overtime, for example. When the light emission characteristic of the light emitting element 10 is changed overtime, an amount of the light received by the first light receiving element 52 changes. When the light emission characteristic of the light emitting element 10 is deteriorated, an amount of the light received by the first light receiving element 52 decreases. Thus, the concentration measurement unit 50 may measure the concentration of the measurement target gas 90 to be higher than the actual concentration. When the light emission characteristic of the light emitting element 10 is enhanced through aging or the like, an amount of the light received by the first light receiving element 52 increases. Thus, the concentration measurement unit 50 may measure the concentration of the measurement target gas 90 to be lower than the actual concentration. By calibrating, by the concentration measurement unit 50, the light receiving characteristic of the first light receiving element 52 with the light receiving characteristic of the second light receiving element 55, and measuring the concentration of the measurement target gas 90 based on the calibrated light receiving characteristic, even when the light emission characteristic of the light emitting element 10 is changed overtime, the concentration measurement unit 50 may accurately measure the concentration of the measurement target gas 90.

The intensity Ir of the light 12 may be a ratio between a first difference between the first intensity and the third intensity of the light 12 received by the first light receiving element 52 and a second difference between the first intensity and the third intensity of the light 12 received by the second light receiving element 55. The intensity Ir of the light 12 may be a difference between the first difference and the second difference. The intensity Ir of the light 12 may be a ratio between a third difference between the second intensity and the third intensity of the light 12 received by the first light receiving element 52 and a fourth difference between the second intensity and the third intensity of the light 12 received by the second light receiving element 55. The intensity Ir of the light 12 may be a difference between the third difference and the fourth difference.

The concentration measurement unit 50 may measure a concentration D1 of the measurement target gas 90 based on the first intensity of the third intensity of the light 12, and measure a concentration D2 of the measurement target gas 90 based on the second intensity and the third intensity of the light 12. The concentration measurement unit 50 may measure the concentration of the measurement target gas 90 based on the concentration D1 and the concentration D2. For example, the concentration measurement unit 50 may measure an average value of the concentration D1 and the concentration D2 as the concentration of the measurement target gas 90. The concentration D1 and the concentration D2 are concentrations measured in cases where direct current power with different polarities are respectively supplied. Thus, a concentration based on the concentration D1 and the concentration D2 may be a more accurate concentration than a concentration obtained by subtracting the third concentration from the first concentration or a concentration obtained by subtracting the third concentration from the second concentration. The storage unit 54 of the concentration measurement unit 50 may store, for each polarity of the direct current power of the light emitting element 10, a calibration curve indicating a relationship between the intensity of light and the concentration, as illustrated in FIG. 17. The concentration measurement unit 50 may select the calibration curve according to the polarity of the direct current power of the light emitting element 10 when the light 12 is received by the first light receiving element 52. The concentration measurement unit 50 may calculate the concentration of the measurement target gas from the intensity of the light 12, based on the selected calibration curve.

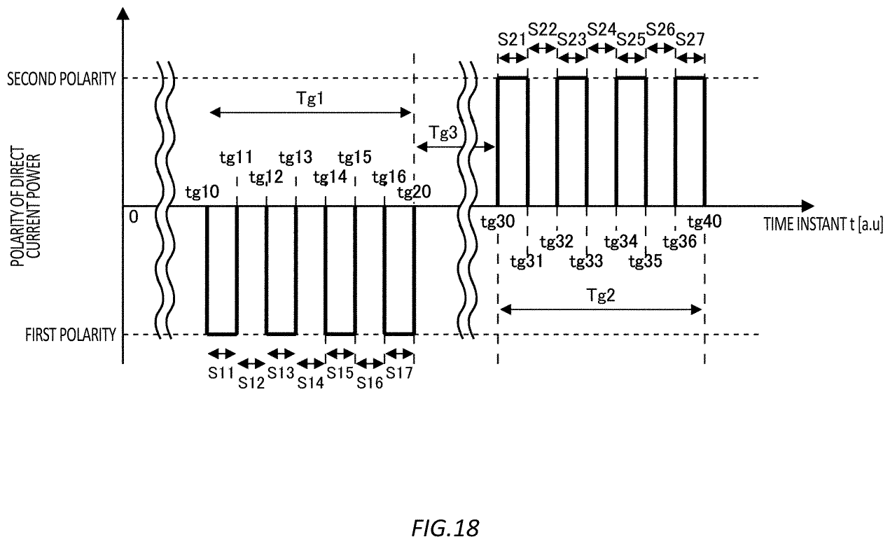

FIG. 18 illustrates another example of the relationship between the polarity of the direct current power and the time instant t. A time instant tg10, a time instant tg20, a time instant tg30, and a time instant tg40 in the present example are the same as the time instant tg1, the time instant tg2, the time instant tg3, and the time instant tg4 in FIG. 16, respectively. In the present example, during a period S11 which is between the time instant tg10 and a time instant tg11, a period S13 which is between a time instant tg12 and a time instant tg13, a period S15 which is between a time instant tg14 and a time instant tg15, and a period S17 which is between a time instant tg16 and a time instant tg20 in the first period Tg1, the first terminal Ev1 (see FIG. 2 and FIG. 3) is maintained at the first potential V1 and the second terminal Ev2 (see FIG. 2 and FIG. 3) is maintained at the second potential V2. Each of the period S11, the period S13, the period S15, and the period S17 is an example of the first measurement period. During the first measurement period, an absolute value of the potential difference between the first terminal Ev1 and the second terminal Ev2 is maintained at a first value or more. Although, in the example described above, the absolute value of said potential difference is maintained at a constant |V1−V2|, the absolute value of said potential difference may not be maintained at a constant value. The first value may be, for example, 2V, may be 3V, or may be a value of 4V or more. As illustrated in FIG. 18, the first measurement period is repeated for two or more times in one first period Tg1. In the present example, during a period S12 which is between the time instant tg11 and the time instant tg12, a period S14 which is between the time instant tg13 and the time instant tg14, and a period S16 which is between the time instant tg15 and the time instant tg16 in the first period Tg1, the direct current power is not supplied. The period S12, the period S14, and the period S16 are examples of the third measurement period. As illustrated in FIG. 18, the third measurement period is a period that is different from the first measurement period. In the present example, in the first period Tg1, the first measurement period and the third measurement period are alternately repeated. The lengths of the period S11 to the period S17 may be the same as one another, or may be different from one another. During the third measurement period, a third value which is an absolute value of the potential difference between the first terminal Ev1 and the second terminal Ev2 is maintained at 0V or more and less than the first value. The third value may be maintained at 0V, or may be maintained at another value. The third value may be half or less of the first value. The third value may be a value of 1V or less, or may a value of 0.5V or less.

In the present example, during a period S21 which is between a time instant tg30 and a time instant tg31, a period S23 which is between a time instant tg32 and a time instant tg33, a period S25 which is between a time instant tg34 and a time instant tg35, and a period S27 which is between a time instant tg36 and a time instant tg40 in the second period Tg2, the first terminal Ev1 (see FIG. 2 and FIG. 3) is maintained at the first potential V1, and the second terminal Ev2 (see FIG. 2 and FIG. 3) is maintained at the second potential V2. Each of the period S21, the period S23, the period S25, and the period S27 is an example of the first measurement period. In the present example, during a period S22 which is between the time instant tg31 and the time instant tg32, a period S24 which is between the time instant tg33 and the time instant tg34, and a period S26 which is between the time instant tg35 and the time instant tg36 in the second period Tg2, the direct current power is not supplied. The period S22, the period S24, and the period S26 are examples of the third measurement period. In the present example, in the second period Tg2, the first measurement period and the third measurement period are alternately repeated. The lengths of the period S21 to the period S27 may be equal to one another, or may be different from one another.

The timing when each of the first measurement periods starts is defined as a first timing. The starting time instant tg10, tg12, tg14, and tg16 of the period S11, the period S13, the period S15, and the period S17 are examples of the first timing. The starting time instant tg30, tg32, tg34, and tg36 of the period S21, the period S23, the period S25, and the period S27 are also examples of the first timing.

The timing when each of the third measurement periods starts is defined as a third timing. The starting time instant tg11, tg13, and tg15 of the period S12, the period S14, and the period S16 are examples of the third timing. The starting time instant tg31, tg33, and tg35 of the period S22, the period S24, and the period S26 are also examples of the third timing.

In the first period Tg1 or the second period Tg2, the first timing and the third timing are alternately arranged. Each of the first measurement periods may start at the first timing and end at the third timing. Also, each of the third measurement periods may start at the third timing and end at the first timing. That is, the first measurement period and the third measurement period arranged alternately may be continuous with each other.

In the present example, the power supply unit 20 supplies the direct current power with the first polarity in the first period Tg1, and supplies the direct current power with the second polarity in the second period Tg2. In the third period Tg3, similarly to the example of FIG. 16, the first terminal Ev1 (see FIG. 2 and FIG. 3) may be maintained at the first potential V1, and the second terminal Ev2 (see FIG. 2 and FIG. 3) may be maintained at the first potential V1 or more and less than the second potential V2.

The concentration measurement unit 50 may measure the concentration of the measurement target gas 90 based on the first intensity of the light 12 received by the first light receiving element 52 in any period of the period S11, the period S13, the period S15, or the period S17 and the third intensity of the light 12 received by the first light receiving element 52 in any period of the period S12, the period S14, or the period S16. The concentration measurement unit 50 may measure the concentration of the measurement target gas 90 based on the first intensity of the light 12 received by the first light receiving element 52 in any period of the period S21, the period S23, the period S25, or the period S27 and the third intensity of the light 12 received by the first light receiving element 52 in any period of the period S22, the period S24, or the period S26. Similarly to the example of FIG. 16, the concentration measurement unit 50 may calculate the concentration of the measurement target gas 90 based on the difference between the first intensity and the third intensity of the light 12, or may calculate the concentration of the measurement target gas 90 based on the ratio between the first intensity and the third intensity of the light 12.

The time instant tg10 in the present example is, for example, a timing of the key switch being turned ON or a timing of the push start for the 2m-1^{th} time described above. The time instant tg30 in the present example is, for example, the timing of the key switch being turned ON or the timing of the push start for the 2m^{th} time described above. Third period Tg3 in the present example is, for example, a period during which the key switch is turned OFF, or the push start is not performed.

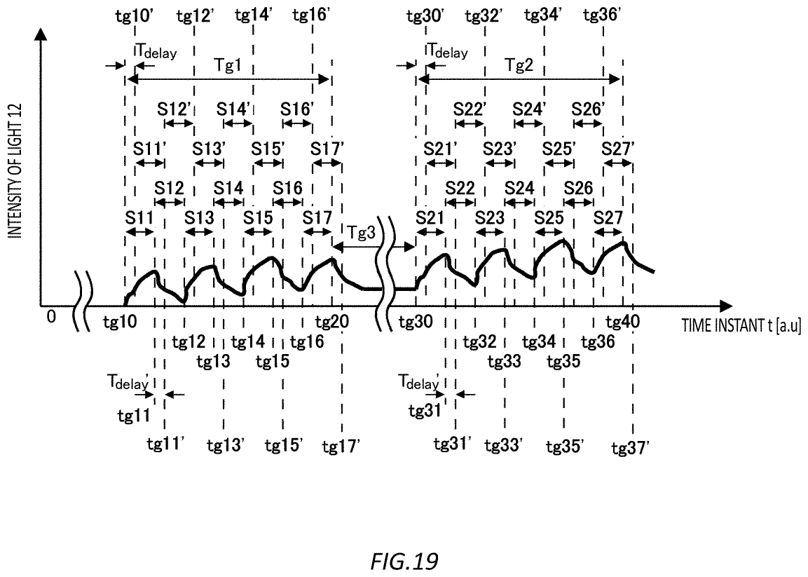

FIG. 19 illustrates an example of a relationship between an intensity of the light 12 received by the first light receiving element 52 and a time instant t. The period S21 to the period S27 in the period S11 to S17 are the same as those in the example of FIG. 18. Periods, which are the period S11 to the period S17 and the period S21 to the period S27 delayed by a predetermined period T_{delay}, are defined as a period S11′ to a period S17′ and a period S21′ to a period S27′, respectively. The period T_{delay} may have a length of 5% or more of each period (for example, each of the period S11 to the period S17, the period S21 to the period S27), may have a length of 25% or more thereof, or may have a length of 50% or more.

Each of the period S11′, the period S13′, the period S15′, and the period S17′ in the first period Tg1 are examples of the second measurement period. Each of the second measurement periods starts at a second timing, which is later than a first timing (for example, the time instant tg10) at which the first measurement period starts and before a third timing (for example, the time instant tg11) at which the third measurement period starts. In the example of FIG. 19, the time instant tg10′, the time instant tg12′, the time instant tg14′, the time instant tg16′ corresponds to the second timing.

In the first period Tg1, each of the period S12′, the period S14′, the period S16′ is an example of a fourth measurement period. Each of the fourth measurement periods starts at a fourth timing after the second measurement period has ended, and which is later than the third timing (for example, the time instant tg11)and before a next occurrence of the first timing (for example, the time instant tg12) at which the first measurement period starts again. In the example of FIG. 19, the time instant tg11′, the time instant tg13′, and the time instant tg15′ correspond to the fourth timing.

In the second period Tg2, each of the period S21′, the period S23′, the period S25′, and the period S27′ is an example of the second measurement period. Also, the time instant tg30′, the time instant tg32′, the time instant tg34′, and the time instant tg36′ correspond to the second timing. Each of the period S22′, the period S24′, and the period S26′ is an example of the fourth measurement period. Also, the time instant tg31′, the time instant tg33′, and the time instant tg35′ correspond to the fourth timing.

In the first period Tg1 or the second period Tg2, the second timing and the fourth timing are alternately arranged. In the example of FIG. 19, in the first period Tg1 or the second period Tg2, the first timing, the second timing, the third timing, and the fourth timing are repeated for two times or more in this order. Each of the second measurement periods may start at the second timing and end at the fourth timing. Also, each of the fourth measurement periods may start at the fourth timing and end at the second timing. That is, the second measurement period and the fourth measurement period that are alternately arranged may be continuous with each other.

The concentration measurement unit 50 may measure the concentration of the measurement target gas 90 based on the second intensity of the light received by the first light receiving element 52 in the second measurement period (for example S11′) that starts at the second timing (for example, tg10′) and the fourth intensity of the light received by the first light receiving element 52 in the fourth measurement period (for example, S12′) that starts at the fourth timing (for example tg11′). For example, the concentration measurement unit 50 calculates the concentration of the measurement target gas 90 based on the difference between the second intensity and the fourth intensity (the second intensity—the fourth intensity). The concentration measurement unit 50 may calculate the concentration of the measurement target gas 90 based on the ratio between the second intensity and the fourth intensity (the second intensity/the fourth intensity).

The concentration measurement unit 50 of the present example measures the concentration of the measurement target gas 90 based on the second intensity of the light 12 received by the first light receiving element 52 in any period of the period S11′, the period S13′, the period S15′, or the period S17′ and the fourth intensity of the light 12 received by the first light receiving element 52 in any period of the period S12′, the period S14′, or the period S16′. The concentration measurement unit 50 of the present example may measure the concentration of the measurement target gas 90 based on the second intensity of the light 12 received by the first light receiving element 52 in any period of the period S21′, the period S23′, the period S25′, or the period S27′ and the fourth intensity of the light 12 received by the first light receiving element 52 in any period of the period S22′, the period S24′, or the period S26′.

Even when the direct current power with the first polarity starts being supplied at the time instant tg10, there may be a delay time until the amount of light of the filament 14 is increased to a desired amount of light or temperature. This delay time is defined as a first delay time. The first desired amount of light or temperature is an amount of light or temperature that each correspond to a value of the direct current power, for example. The period T_{delay} may be equal to the first delay time. The same applies at the time instant tg12, the time instant tg14, the time instant tg16, the time instant tg30, the time instant tg32, the time instant tg34, and the time instant tg36.

Even when supply of the direct current power with the first polarity starts being stopped at the time instant tg11, there may be a delay time until the amount of light of the filament 14 is decreased to a desired amount of light or temperature. This delay time is defined as a second delay time. The desired amount of light or temperature is an amount of light or temperature that each correspond to a value of the direct current power being zero, for example. The period T_{delay} may be equal to the second delay time. The same applies at the time instant tg13, the time instant tg15, the time instant tg17, the time instant tg31, the time instant tg33, the time instant tg35, and the time instant tg37. The period T_{delay}′ may be different from or equal to the period T_{delay}.

When there is a first delay time until the amount of light of the filament 14 is increased to the desired amount of light or there is a second delay time until it is decreased to the desired amount of light, the concentration measurement unit 50 may measure the concentration of the measurement target gas 90 based on the first intensity of the light 12 received by the first light receiving element 52 in any period of the period S11′, the period S13′, the period S15′, or the period S17′ and the third intensity of the light 12 received by the first light receiving element 52 in any period of the period S12′, the period S14′, or the period S16′. The concentration measurement unit 50 may thereby more accurately measure the concentration of the measurement target gas 90.

In the first period Tg1 or the second period Tg2, the first measurement period may be repeated for two or more times at a frequency of 0.1 Hz or more. In the example of FIG. 19, in the first period Tg1, the first measurement period is repeated four times (the period S11, the period S13, the period S15, and the period S17) at a frequency of 0.1 Hz or more. In the second period Tg2, the first measurement period is also repeated four times (the period S21, the period S23, the period S25, and the period S27) at a frequency of 0.1 Hz or more. The repetition frequency of the first measurement period may be 0.5 Hz or more, may be 1 Hz or more, or may be 10 Hz or more. Said repetition frequency may be 100 Hz or less.

In the sensing of the measurement target gas, the shorter the drive cycle of the gas sensor 100, the more accurately the variation in the gas concentration can be measured. As an example, the gas sensor 100 that senses alcohol, the drive cycle is preferably short. On the other hand, when the drive cycle of the gas sensor 100 becomes shorter, the light emittance of the filament of the light emitting element 10 becomes less stable. As described above, in the example of FIG. 18 and FIG. 19, the measuring timing by the first light receiving element 52 (the second timing) is delayed relative to the light emittance starting timing of the light emitting element 10 (the first timing). Thus, the concentration measurement unit 50 can more accurately measure the concentration of the measurement target gas 90.

The gas sensor 100 may wait until the light emittance by the light emitting element 10 is stabilized to perform measurement by the first light receiving element 52. The gas sensor 100 may set each delay time described above such that the light emittance by the light emitting element 10 is stabilized.

The gas sensor 100 may set each delay time described in FIG. 19 to 0. That is, the third timing described above may be matched with the first timing and the fourth timing may be matched with the second timing. In this case, the concentration measurement unit 50 may measure the concentration of the measurement target gas 90 based on the first intensity of the light received by the first light receiving element 52 in the first measurement period (for example, S11) and the third intensity of the light received by the first light receiving element 52 in the third measurement period (for example, S12).

In each example described in the present specification, the first period Tg1, the second period Tg2, and the third period Tg3 of the first light receiving element 52 and the second light receiving element 55 may be the same. On the other hand, each period, each time instant, and each delay time included in the first period Tg1, the second period Tg2, and the third period Tg3 may different for the first light receiving element 52 and the second light receiving element 55. For example, the length of at least one of the second measurement period (for example, the period S11′, the period S13′, the period S15′, and the period S17′) or the fourth measurement period (for example, the period S12′, he period S14′, and the period S16′) in the example of FIG. 19 may be different for the first light receiving element 52 and the second light receiving element 55. At least one of the second timing at which the second measurement period starts or the fourth timing at which the fourth measurement period starts may be different for the first light receiving element 52 and the second light receiving element 55. The first optical filter 51 of the first light receiving element 52 and the second optical filter 53 of the second light receiving element 55 may be different in the band that passes therethrough.

The light emittance spectrum changes according to the temperature of the filament of the light emitting element 10. Since the wavelength bands detected by the first light receiving element 52 and the second light receiving element 55 are different, the delay time until the light emittance at the light emitting element 10 or the output current is different. Thus, the first light receiving element 52 and the second light receiving element 55 may have different optimal settings for the parameters in each period, each time instant, and each delay time included in the first period Tg1, the second period Tg2, and the third period Tg3. By adjusting these parameters for each light receiving element, the concentration of the measurement target gas 90 can be more accurately measured by each light receiving element.

For example, an infrared absorption wavelength of alcohol is near 3.3 um and an absorption wavelength of carbon dioxide is near 4.3 um, so each light receiving element is designed in accordance thereto. The light emittance spectrum characteristics of the filament of the light emitting element 10 is dependent on the temperature of the filament. Compared to alcohol, carbon dioxide may be detected based on light emittance when the temperature of the filament relatively low.

When the measurement target gas 90 is carbon dioxide, the first light receiving element 52 may detect carbon dioxide, and the second light receiving element 55 may detect a reference gas. The reference gas may be a gas with a known concentration or a concentration with approximately no change. By adjusting the parameters described above for the first light receiving element 52 and the second light receiving element 55, the concentration of carbon dioxide can be accurately measured.

The measurement target gas 90 may be alcohol and carbon dioxide. For example, the gas sensor 100 may be used to sense a drinking state of a subject. The first light receiving element 52 may detect alcohol, and the second light receiving element 55 may detect carbon dioxide. By adjusting the parameters described above for the first light receiving element 52 and the second light receiving element 55, the concentration can be accurately measured for each of alcohol and carbon dioxide. Thus, sensing accuracy of the drinking state can be enhanced.

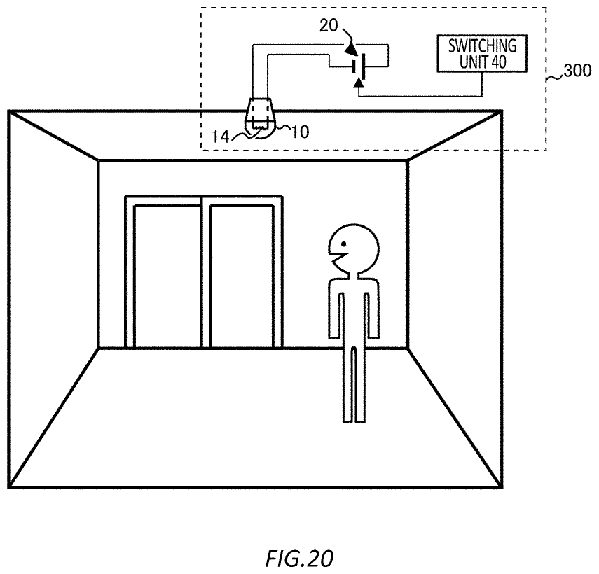

FIG. 20 illustrates an example of an power supply system 300 according to one embodiment of the present invention. In the present example, the light emitting element 10 is a lightbulb provided in a room.

The power supply system 300 includes a power supply unit 20 and a switching unit 40. The power supply system 300 may include a light emitting element 10. The power supply unit 20 supplies direct current power to the light emitting element 10. The light emitting element 10 is of a thermal radiation type having a filament 14. The switching unit 40 switches the polarity of the direct current power based on a set condition. The light emitting element 10, the power supply unit 20, and the switching unit 40 may be connected as illustrated in the circuit diagram shown in FIG. 2 or FIG. 3. Operations that are similar to the operations described with the example of the gas sensor 100 in FIG. 4 to FIG. 19 may be achieved in the power supply system 300.

The light emitting element 10 may be a lightbulb used for a warning light on the road or in a building. The power supply unit 20 in the power supply system 300 may supply direct current power to such a light emitting element 10. The switching unit 40 in the power supply system 300 may switch the polarity of the direct current power based on a set condition.

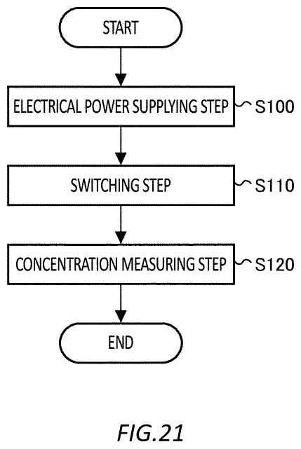

FIG. 21 is a flowchart illustrating an example of a measurement method of a gas concentration according to one embodiment of the present invention. The measurement method of the gas concentration is described with the example of the gas sensor illustrated in FIG. 1A to FIG. 19. The measurement method of the gas concentration includes an electrical power supplying step S100, a switching step S110, and a concentration measuring step S120.

The electrical power supplying step S100 is a step of supplying, by the power supply unit 20, direct current power to the light emitting element 10 of a thermal radiation type having the filament 14. The light emitting element 10 emits light 12 to be radiated to the measurement target gas 90. The switching step S110 is a step of switching, by the switching unit 40, the polarity of the direct current power based on a set condition. The concentration measuring step S120 is a step of measuring the concentration of the measurement target gas 90 to which the light 12 is radiated.

While the present invention has been described by way of the embodiments, the technical scope of the present invention is not limited to the above-described embodiments. It is apparent to persons skilled in the art that various alterations or improvements can be made to the above described embodiments. It is also apparent from description of the claims that the embodiments to which such modifications or improvements are made may be included in the technical scope of the present invention.

It should be noted that each process of the operations, procedures, steps, stages, and the like performed by the apparatus, system, program, and method shown in the claims, specification, or drawings can be executed in any order as long as the order is not indicated by “prior to”, “before”, or the like and as long as the output from a previous process is not used in a later process. Even if the operation flow is described using phrases such as “first” or “next” for the sake of convenience in the claims, specification, or drawings, it does not necessarily mean that the process must be performed in this order.

What is claimed is:

1. A breath sensor comprising:
a light source unit that has a light emitting element of a thermal radiation type including a filament and radiates light toward a measurement target gas related to breath; and
a concentration measurement unit that has a light receiving element which receives at least a part of the light, and that measures a concentration of the measurement target gas, wherein
the light source unit has a first terminal and a second terminal,
the light emitting element is supplied, from the first terminal and the second terminal, with direct current power of which a polarity is switched based on a set condition,
in a first measurement period during which the direct current power is supplied to the light emitting element, the first measurement period being repeated for two or more times, an absolute value of a potential difference between the first terminal and the second terminal is maintained at a first value or more,
in a third measurement period that is different from the first measurement period, a third value, which is an absolute value of a potential difference between the first terminal and the second terminal is maintained to be 0V or more and less than the first value, and
the concentration measurement unit measures a concentration of the measurement target gas based on:
a second intensity of the light received by the light receiving element during a second measurement period which starts at a second timing which is later than a first timing at which the first measurement period starts and before a third timing at which the third measurement period starts; and
a fourth intensity of the light received by the light receiving element during a fourth measurement period which starts at a fourth timing after the second measurement period has ended, and which is later than the third timing and before a next occurrence of the first timing at which the first measurement period starts again.

2. The breath sensor according to claim 1, wherein
the first measurement period is repeated for two or more times at a frequency of 0.1 Hz or more.

3. The breath sensor according to claim 1, wherein switching is performed between a first polarity, which is one of the polarities, and a second polarity which is another of the polarities, based on a number of times of activation of the breath sensor.

4. The breath sensor according to claim 1, wherein switching is performed between a first polarity, which is one of the polarities, and a second polarity, which is another of the polarities, based on a number of times of activation of the light emitting element.

5. The breath sensor according to claim 1, wherein
switching is performed between a first polarity, which is one of the polarities, and a second polarity, which is another of the polarities, based on a supply time of the direct current power.

6. The breath sensor according to claim 1, wherein
switching is performed between a first polarity, which is one of the polarities, and a second polarity, which is another of the polarities, based on an activation time of the breath sensor.

7. The breath sensor according to claim 1, further comprising:
a resistance acquisition unit that acquires a resistance value of the light emitting element, and
switching is performed between a first polarity, which is one of the polarities, and a second polarity, which is another of the polarities, based on the resistance value.

8. The breath sensor according to claim 1, wherein switch is performed between a first polarity, which is one of the polarities, and a second polarity, which is another of the polarities, based on a temperature or humidity of a measurement target including the measurement target gas.

9. The breath sensor according to claim 1, wherein
the direct current power is supplied by a constant-voltage power supply,
the constant-voltage power supply has the first terminal and the second terminal,
the constant-voltage power supply maintains a potential of the first terminal at a first potential, maintains a potential of the second terminal at a second potential, which is a higher potential than the first potential, and supplies the direct current power through a potential difference between the first potential and the second potential, and
the constant-voltage power supply increases the potential difference in a stepwise manner.

10. The breath sensor according to claim 1, wherein
the direct current power is supplied by a constant-current power supply.

11. The breath sensor according to claim 1, wherein
a concentration of the measurement target gas measured in a period of a first polarity, which is one of the polarities, is defined as a first gas concentration, and a concentration of the measurement target gas measured in a period of second polarity, which is another of the polarity, is defined as a second gas concentration, and
the concentration measurement unit corrects at least one of the first gas concentration or the second gas concentration, based on a first gas concentration measured during a period of the first polarity and a second gas concentration measured during a period of the second polarity, to calculate a concentration of the measurement target gas.

12. The breath sensor according to claim 1, wherein
the concentration measurement unit stores, for each polarity of the direct current power of the light emitting element, a calibration curve indicating a relationship between an intensity of the light and the concentration, and calculates a concentration of the measurement target gas based on the calibration curve according to the polarity.

13. The breath sensor according to claim 1, comprising two or more light receiving elements, each being identical to the light receiving element, wherein
in the concentration measurement unit, at least one of lengths of the second measurement period or the fourth measurement period is different for each of the light receiving elements.

14. The breath sensor according to claim 1, comprising two or more light receiving elements, each being identical to the light receiving element, wherein
in the concentration measurement unit, at least one of the second timing, which is a starting time point of the second measurement period, or the fourth timing, which is a starting time point of the fourth measurement period, is different for each of the light receiving elements.

15. A measurement method of breath comprising:
supplying direct current power to a light source unit that includes a light emitting element of a thermal radiation type having a filament and has a first terminal and a second terminal, wherein the light emitting element emits light to be radiated to a measurement target gas related to breath; and
measuring a concentration of the measurement target gas to which the light is radiated, by using a light receiving element that receives at least a part of the light;
wherein a direct current power of which a polarity is switched based on a set condition is supplied to the light emitting element from the first terminal and the second terminal,
in a first measurement period during which the direct current power is supplied to the light emitting element, the first measurement period being repeated for two or more times, an absolute value of a potential difference between the first terminal and the second terminal is maintained at a first value or more,
in a third measurement period that is different from the first measurement period, a third value, which is an absolute value of a potential difference between the first terminal and the second terminal is maintained to be 0V or more and less than the first value, and
a concentration of the measurement target gas is measured based on:
a second intensity of the light received by the light receiving element during a second measurement period which starts at a second timing that is later than a first timing at which the first measurement period starts and before a third timing at which the third measurement period starts; and
a fourth intensity of the light received by the light receiving element during a fourth measurement period which starts at a fourth timing after the second measurement period has ended and which is later than the third timing and before a next occurrence of the first timing at which the first measurement period starts again.

16. The measurement method according to claim 15, wherein
the first measurement period is repeated for two or more times at a frequency of 0.1 Hz or more.

17. The measurement method according to claim 16, wherein switching is performed between a first polarity, which is one of the polarities, and a second polarity which is another of the polarities, based on a number of times of activation of a breath sensor that measures a concentration of the measurement target gas.

18. A breath sensor comprising:
a light source unit that has a light emitting element of a thermal radiation type including a filament and radiates light toward a measurement target gas related to breath; and
a concentration measurement unit that has a light receiving element which receives at least a part of the light, and that measures a concentration of the measurement target gas;
wherein the light source unit has a first terminal and a second terminal,
the light emitting element is supplied, from the first terminal and the second terminal, with direct current power of which a polarity is switched based on a set condition,
in a first measurement period during which the direct current power is supplied to the light emitting element, the first measurement period being repeated for two or more times, an absolute value of a potential difference between the first terminal and the second terminal is maintained at a first value or more,
in a third measurement period that is different from the first measurement period, a third value, which is an absolute value of a potential difference between the first terminal and the second terminal is maintained to be 0V or more and less than the first value, and
the concentration measurement unit measures a concentration of the measurement target gas based on:
a first intensity of the light received by the light receiving element in the first measurement period; and
a third intensity of the light received by the light receiving element in the third measurement period.

19. The breath sensor according to claim 18, wherein
the first measurement period is repeated for two or more times at a frequency of 0.1 Hz or more.

20. The breath sensor according to claim 18, wherein switching is performed between a first polarity, which is one of the polarities, and a second polarity which is another of the polarities, based on a number of times of activation of the breath sensor.
